(19) United States
(12) Patent Application Publication
(10) Pub. No.: US 2022/0040434 A1
(43) Pub. Date: Feb. 10, 2022

(54) Fresh Air/Oxygen Portable Breathing Device With or Without Aromas and Methods of Use

(71) Applicant: Hae Dong Jho, Wexford, PA (US)

(72) Inventor: Hae Dong Jho, Wexford, PA (US)

(21) Appl. No.: 16/984,759

(22) Filed: Aug. 4, 2020

(51) Int. Cl. A61M 16/06; A61M 16/20; A61M 16/10; A61M 16/16

(52) U.S. Cl. CPC A61M 16/0672; A61M 16/0605; A61M 2202/0208; A61M 16/1005; A61M 16/16; A61M 16/20

(57) ABSTRACT

The present invention provides a portable, light-weight, breathing device and methods of use for delivering pristine, fresh air or varying high concentrations of oxygen with or without aroma, i.e., essential oils for stimulating appetite, boosting mood or decreasing stress and anxiety. The breathing device comprises an air container having a breathing air bag therein which contains the fresh air or the varying concentrations of oxygen optionally with the one or more essential oils, tubing for delivering the fresh air or oxygen to a breathing mask, and a plurality of movable valves included in the mask which directs air or oxygen into or out of the mask depending on whether the user is inhaling or exhaling. The breathing device also comprises the use of a can of compressed fresh air or oxygen with or without aroma to be used with an air reservoir or with an expandable air bag.

### FIELD OF THE INVENTION

The present invention relates to breathing devices and, in particular, to a portable, lightweight, fresh air or oxygen breathing device for delivering fresh air or varying high concentrations of oxygen to a user, in which aromas optionally are present.

### BACKGROUND OF THE INVENTION

Breathing fresh air outdoors does more than just feel good—it has been shown that it can have surprising health benefits that one cannot get when breathing indoor air. Indoor air often has a suboptimal balance of oxygen, carbon dioxide and nitrogen, which is further decreased in enclosed rooms with poor ventilation. In contrast, fresh air generally has higher levels of oxygen than indoor air. Exposure to more oxygen causes blood vessels in lungs to dilate, which improves cleansing and tissue repair of lung tissue, resulting in more efficient gas exchange.

In addition, breathing fresh air enhances physical energy and mental focus because as lungs take in more fresh air, oxygen levels in the body go up and thus more oxygen circulates through the brain. This facilitates a feeling of energy and improves one's memory and ability to concentrate. Another surprising benefit of breathing fresh air containing optimal levels of oxygen is the promotion of the production of serotonin, which can increase feelings of happiness and decrease anxiety. Other advantages of breathing fresh air with optimal levels of oxygen is the lowering of blood pressure and heart rate; faster healing from illness and injuries; and improved digestion.

Still another surprising benefit of breathing fresh air or different levels of pure oxygen is, when combined with various aromas emanating from one or more essential oils, referred to aromatherapy, inhalation of the fresh air or oxygen can stimulate appetite, boost mood or calm stress and anxiety.

During this unprecedented time when the whole world is experiencing a novel coronavirus pandemic, where millions of people are suffering from varying degrees of compromised lung function, along with physical, emotional and cognitive deficits from the COVID-19 infection (the disease caused by the virus), there is an urgent need to provide a device and method that can, for a relatively short but invaluable period of time, ameliorate the harmful effects experienced by those afflicted with compromised breathing.

### SUMMARY OF THE INVENTION

The present invention fulfills this need by providing a portable, light-weight breathing device with or without aroma and methods of use for delivering pristine, fresh air or varying high concentrations of oxygen to a user, in order to enhance both physiological functioning but also psychological well-being, mood and appetite.

In an aspect of the invention, there is provided a portable breathing device comprised of an air container having an interior, an opening on its top surface and an outer cap which fits over the opening of the air container; a breathing air bag located in the interior of the air container and having an opening at one end, the breathing air bag opening having an air bag tubing connector affixed thereon; an inner cap having a tubing portal and a plurality of air release holes; a tubing insert having two ends, in which one end fits into the tubing portal; a tubing insert connector having a top end and a bottom end, the bottom end comprised of a split valve; the top end of the tubing insert connector fitting into the other end of the tubing insert; the bottom end of the tubing insert connector with the split valve fitting into the air bag tubing connector. The portable breathing device further comprises a breathing mask having a central opening, a moveable central valve which covers the central opening, a plurality of exhalation holes on both sides of the mask, a pair of moveable exhalation hole valves, each one of the pair of exhalation hole valves adjacent to the exhalation holes in the breathing mask and covering the exhalation holes, and a strap to secure the breathing mask to a user. The breathing device further comprises tubing having a first end and a second end, a mask tubing connector which is affixed at one end within the central opening of the breathing mask and affixed at the other end to the first end of the tubing, with the second end of the tubing inserted into the breathing air bag via the tubing insert, the tubing insert connector and the breathing air bag tubing connector.

The method of using the above-described portable breathing device comprises (a) removing the outer cap from the top of the breathing container; (b) placing one end of the tubing into the breathing air bag by inserting the tubing through the tubing insert, the tubing insert connector, the split valve and the breathing air bag tubing connector; (c) placing the breathing mask over the nose and mouth of a user and using the strap to secure the breathing mask to the user's face; (d) having the user inhale, which moves the central valve upwardly to uncover the central opening, which releases air from the breathing air bag into the tubing and then into the mask so that the user inhales the fresh air or the varying high concentrations of oxygen with or without aroma contained in the breathing air bag; (e) having the user exhale, which moves the central valve downwardly to cover the central opening which stops the entry of air from the breathing air bag into the tubing and into the mask, and moves the exhalation hole valves outwardly to uncover the exhalation holes so that the exhaled air is expelled through the exhalation holes; (f) repeating steps (d) and (e) until all the fresh air or varying high concentrations of oxygen is released from the breathing bag; (g) removing the tubing from the breathing air bag and the mask from the user; and (h) placing the outer cap on the top of the air container.

In another aspect of the invention, there is provided a portable breathing device for delivering fresh air or varying high concentrations of oxygen with or without aroma to a user, comprising a can containing compressed fresh air or varying high concentrations of compressed oxygen with or without aroma, the can having on its top surface a release button, the release button containing a release button air hole, and a compressible release hood which covers the release button, the compressible release hood having an opening therethrough; a breathing mask having a central opening, a moveable central valve which covers the central opening, a plurality of exhalation holes on both sides of the mask, a pair of moveable exhalation hole valves, each one of the pair of exhalation hole valves adjacent to the exhalation holes in the breathing mask to cover the exhalation holes, and a strap to secure the breathing mask to a user; tubing having a first end and a second end; a mask tubing connector affixed to the central opening of the breathing mask, the mask tubing connector affixed to the first end of the tubing and the second end of the tubing affixed into the opening of the compressible release hood; and an inflatable air reservoir positioned on and encircling a length of the tubing, wherein the tubing that is encircled by the inflatable air reservoir has a plurality of tubing air holes to allow air to enter and exit the air reservoir.

The method of using the above-described portable breathing device comprises (a) placing the compressible release hood with affixed tubing over the release button of the can of compressed fresh air or varying high concentrations of oxygen with or without aroma; (b) placing the breathing mask over the nose and mouth of a user and using the strap to secure the breathing mask to the user's face; (c) pressing downwardly on the compressible release hood to release gas into the tubing and into the inflatable air reservoir so that the air reservoir inflates; (d) having the user inhale, which moves the central valve upwardly to uncover the central opening, which pulls air from the air reservoir into the tubing and then into the mask so that the user inhales the fresh air or the varying high concentrations of oxygen with or without aroma contained in the can; (e) having the user exhale, which moves the central valve downwardly to cover the central opening, which stops the entry of air from the air reservoir into the tubing and into the mask, and moves the exhalation hole valves outwardly to uncover the exhalation holes so that the exhaled air is expelled through the exhalation holes; and (f) repeating steps (d) and (e) until all the fresh air or varying high concentrations of oxygen is released from the air reservoir.

In a further aspect of the invention, there is provided a portable breathing device for delivering fresh air or varying high concentrations of oxygen with or without aroma to a user comprising a can containing compressed fresh air or varying high concentrations of compressed oxygen with or without aroma, the can having on its top surface a release button, the release button containing a release button air hole, and a compressible release hood which covers the release button, the compressible release hood having an opening thereon; a breathing mask having a central opening in which one end of a hollow mask interface connector is affixed therein, a moveable central valve which covers the central opening, a plurality of exhalation holes on both sides of the mask, a pair of moveable exhalation hole valves, each one of the pair of exhalation hole valves adjacent to the exhalation holes in the breathing mask covering the exhalation holes, and a strap to secure the breathing mask to a user; a hollow air interface connector having a first end and second end and a plurality of air holes therein, the first end affixed atop the opening of the compressible release hood, the second end affixed to the other end of the mask interface connector; and an expandable air bag having an opening therein, the opening of the expandable air bag affixed to the air interface connector.

The method of using the above-described portable breathing device comprises (a) placing the compressible release hood with affixed tubing over the release button of the can of compressed fresh air or varying high concentrations of oxygen with or without aroma; (b) placing the breathing mask over the nose and mouth of a user and using the strap to secure the breathing mask to the user's face; (c) pressing downwardly on the compressible release hood to release the air or oxygen into the air interface connector and into the expandable air bag through the air holes in the air interface connector; (d) having the user inhale, which moves the central valve upwardly to uncover the central opening which releases air from the expandable air bag into the mask so that the user inhales the fresh air or the varying high concentrations of oxygen with or without aroma contained in the expandable air bag; (e) having the user exhale, which moves the central valve downwardly to cover the central opening which stops the entry of air into the mask from the expandable air bag, and moves the exhalation hole valves outwardly to uncover the exhalation holes so that the exhaled air is expelled through the exhalation holes; and (f) repeating steps (d) and (e) until all the fresh air or varying high concentrations of oxygen is released from the expandable air bag.

### BRIEF DESCRIPTION OF THE DRAWINGS

For the present disclosure to be easily understood and readily practiced, the present disclosure will now be described for purposes of illustration and not limitation in connection with the following figures, where like reference numerals refer to identical or functionally similar elements throughout the separate views, wherein:

FIG. 1 is a front perspective view of the invention, showing the fresh air/oxygen container, the breathing mask, and the tubing connecting the container to the mask, in accordance with embodiments of the present invention;

FIG. 2 a front perspective view of the inner cap, the tubing insert and the tubing insert connector with split valve, in accordance with embodiments of the present invention;

FIG. 3 is a front perspective view of the breathing air bag, in accordance with embodiments of the present invention;

FIG. 4 is a front perspective view of the breathing mask, in accordance with embodiments of the present invention;

FIG. 5 are perspective views of the invention, showing a side perspective view of the breathing mask with attached tubing; and the fresh air/oxygen container with associated components, in accordance with embodiments of the present invention;

FIG. 6 is a side perspective view of the breathing mask with an exhalation hole covered by an exhalation hole valve, in accordance with embodiments of the present invention;

FIG. 7 is a side perspective view of the breathing mask with an exhalation hole uncovered, in accordance with embodiments of the present invention;

FIG. 8 is a rear perspective view of the breathing mask with the opening to the tubing connector covered by the central valve, in accordance with embodiments of the present invention;

FIG. 9 is a rear perspective view of the breathing mask with the opening to the tubing connector uncovered, in accordance with embodiments of the present invention;

FIG. 10 is a front perspective view of the invention, showing the fresh air/oxygen can of compressed air, the air reservoir inflated with air, the breathing mask, and the tubing connecting the can of compressed air to the reservoir and to the mask, in accordance with embodiments of the present invention;

FIG. 11 is a front perspective view of the invention, showing the fresh air/oxygen can of compressed air, the air reservoir deflated, the breathing mask, and the tubing connecting the can of compressed air to the reservoir and to the mask, in accordance with embodiments of the present invention; and

FIG. 12 is a perspective view of the invention showing the can of compressed air closely adjoined to the air reservoir and the breathing mask, in accordance with the embodiments of the present invention.

### DETAILED DESCRIPTION OF THE INVENTION

The present invention provides a portable, light-weight breathing device for breathing pristine, fresh air or varying high concentrations of oxygen with or without aroma by a user.

When used to deliver fresh air or varying high concentrations of oxygen to a user, the invention contemplates that it may be used at any time, at any place and by any user when the user desires or needs the beneficial physical, mental, emotional and/or enhanced cognitive effects attendant with breathing pristine, fresh air. The addition of aroma in the form of essential oils is used to further benefit physical, mental, emotional and/or cognitive effects, as well as to stimulate and enhance appetite.

As used herein, the terms aroma and “essential oil” or “essential oils” are meant to be interchangeable.

As used herein, the terms “fresh air” and “air” are meant to be interchangeable.

As used herein, the terms “oxygen” and “air” are meant to be interchangeable.

As used herein, “fresh air” is defined as air having a humidity factor ranging between about 40% and 60%, and containing the following components in the following percentages: about 21% oxygen; about 78% nitrogen; about 0.9% argon; about 0.03% carbon dioxide; about 0.002% neon; 0.001% helium; about 0.0002% methane; about 0.0001%; krypton; about 0.0001% hydrogen; and about 0.00001% xenon.

Inhalation of varying high concentrations of oxygen will depend on the needs of the user. For example, an individual experiencing cardiac or respiratory arrest may require a percentage of inspired oxygen as high as 100%. In cases of hypoxemia, and depending on the severity of the hypoxemia, the percentage of inspired oxygen an individual may require can range between about 40% and 60%. The invention contemplates that as the percentage of oxygen increases, the corresponding percentage of nitrogen decreases.

The fresh air or the varying concentrations of oxygen optionally contains aromas from one or more essential oils to stimulate appetite, including without limitation, tangerine, cardamom, bergamot, ginger, caraway, roman chamomile, spearmint, peppermint, lavender, and eucalyptus; to boost mood, including without limitation, cinnamon, eucalyptus, frankincense, fresh basil; to alleviate stress and anxiety, including without limitation, lavender, lemon balm, jasmine, rose, chamomile, bergamot, orange and rosemary.

Whether the fresh air or the varying high concentrations of oxygen contain such aromas depends on the needs and/or desires of the user. For example, if a user wants to enhance their dining experience, they may use the fresh air device with fresh air infused with an aroma known to enhance one's appetite as an “appetizer” prior to eating so that their appetite is enhanced and stimulated. As another example, if a user wants to boost their mood, they may use the fresh air device with fresh air infused with an aroma known to boost and improve depressed or sad mood. As a further example, if a user wants to decrease their level of stress or anxiety prior to a stressful event, they may use the fresh air device with fresh air infused with an aroma known to decrease stress and anxiety prior to the stressful event.

The amount of the one or more essential oils which may be added to the fresh air or varying high concentrations of oxygen ranges from about 1 drop to as high as 50 drops or higher per essential oil. The invention contemplates that the amount of the one or more essential oils added to the fresh air or varying high concentrations of oxygen depends on the particular size of the air bag container or the can of compressed air, as well as the particular needs and/or wishes of a user.

A complete understanding of the present invention will be obtained from the following description taken in connection with the accompanying drawing figures, wherein like reference characters identify like parts throughout.

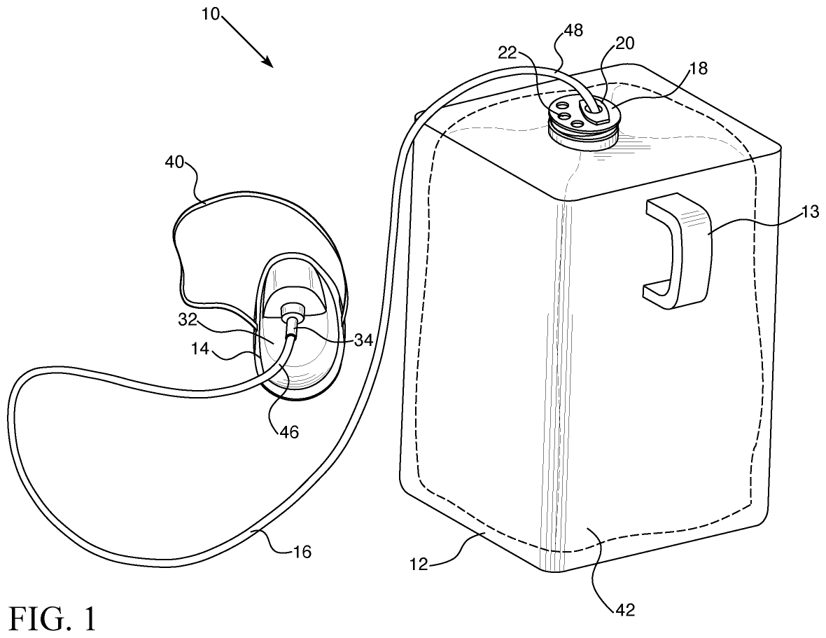

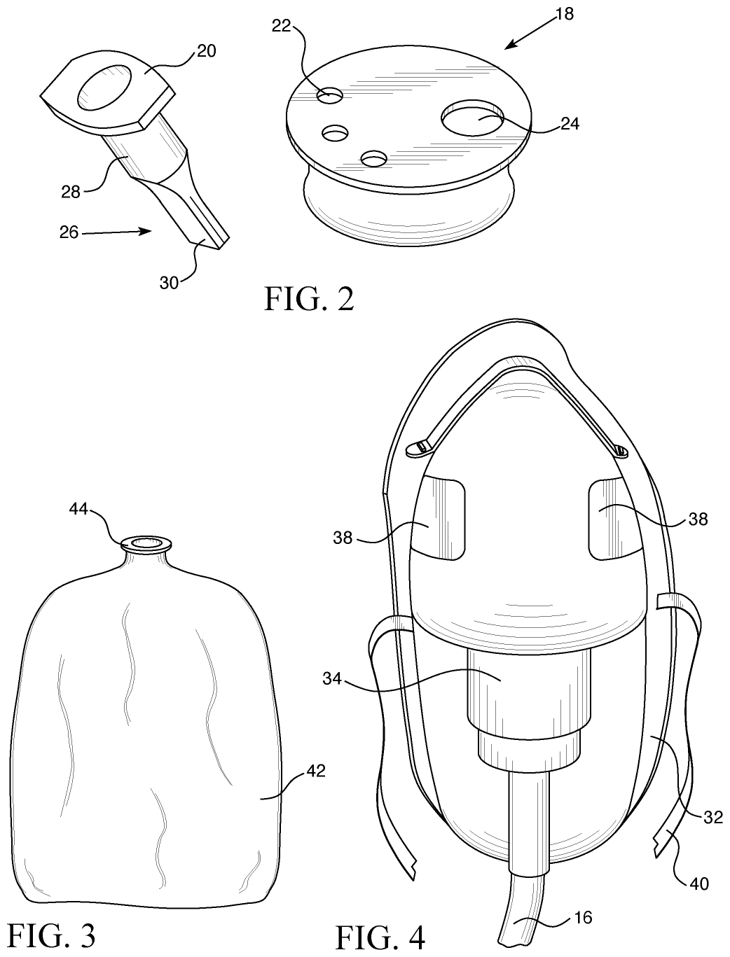

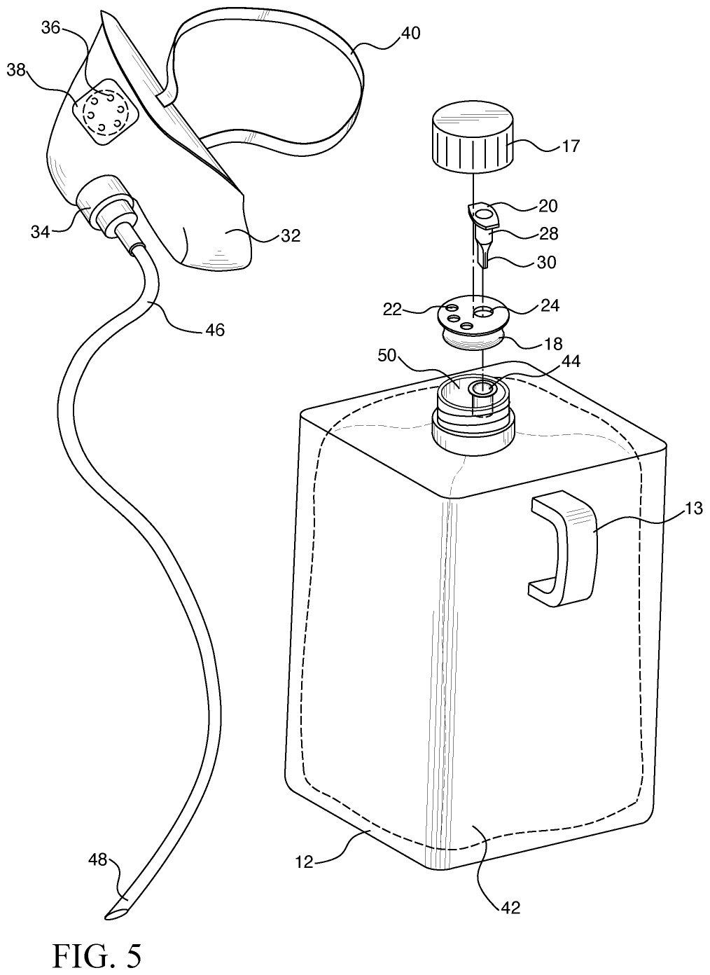

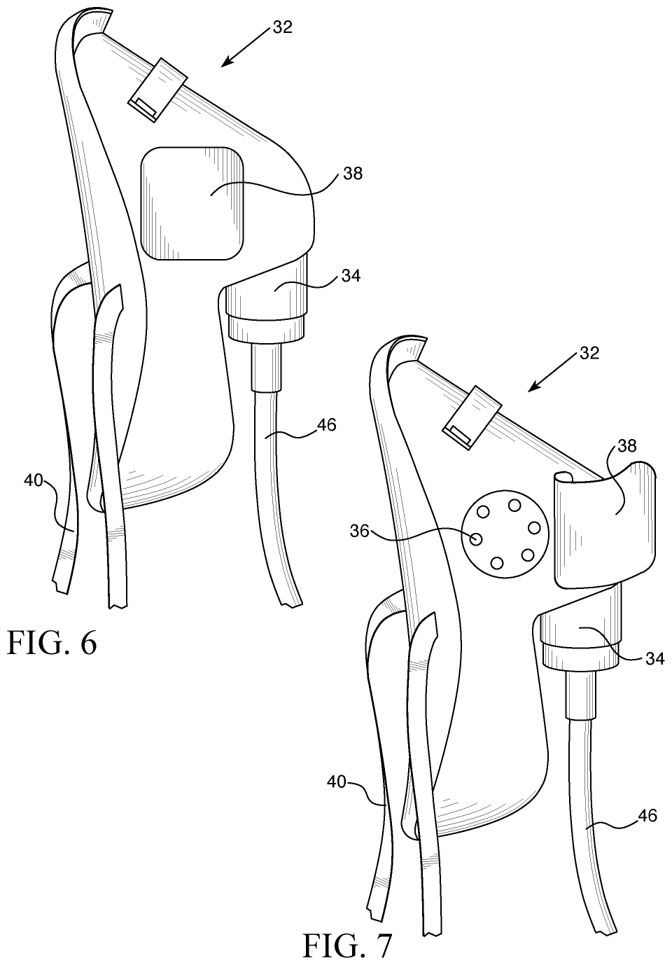

Referring now to FIGS. 1-9, in one embodiment of the invention, as shown generally in FIG. 1, the breathing device 10 for delivering fresh air or varying high concentrations of oxygen with or without aroma to a user comprises an air container 12 having an interior, an opening 50 on its top surface, and an outer cap 17 to cover the opening 50 of the air container 12 (shown in FIG. 5). As shown best in FIGS. 2-5, a breathing air bag 42 is located in the interior of the air container 12. The breathing air bag 42 has an opening at one end in which an air bag tubing connector 44 is affixed thereon. Positioned on the opening 50 of the air container 12 is an inner cap 18 having a tubing portal 24 and a plurality of air release holes 22. One end of a tubing insert 20 fits into the tubing portal 24. A top end 28 of a tubing insert connector 26 fits into the tubing insert 20. At the other end of the tubing insert connector 26 is a split valve 30 which inserts into the breathing air bag 42 via the air bag tubing connector 44. The split valve 30 prevents air from leaking out of the breathing air bag 42 by trapping air within the breathing air bag 42 when the breathing air bag 42 is inflated and not in use.

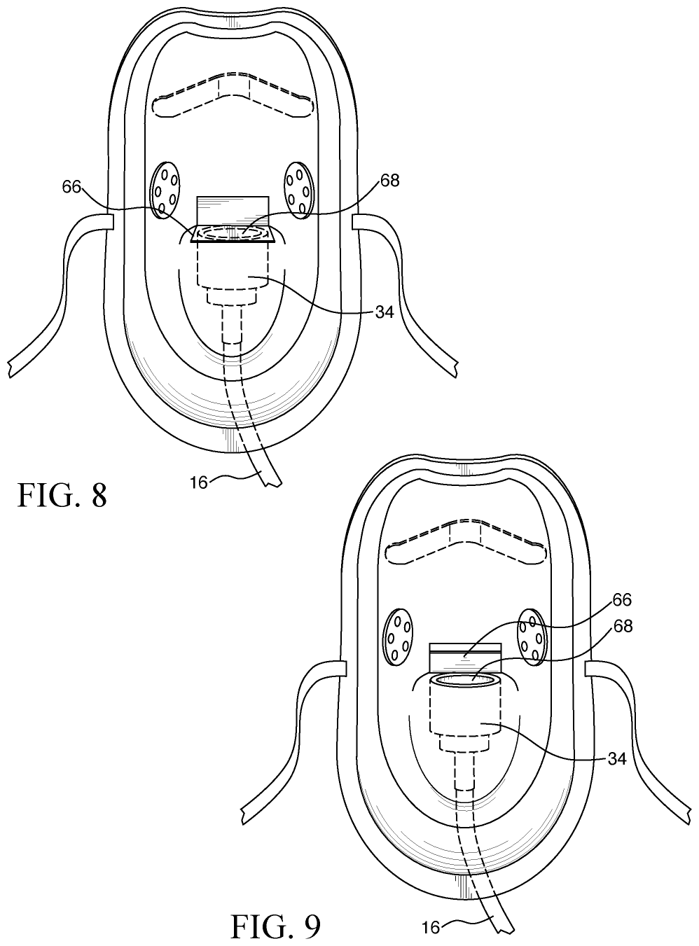

As shown in FIGS. 4, 6 and 7-9, the breathing device 10 also comprises a breathing mask 32. The breathing mask 32 has a central opening 68 and a moveable central valve 66 which covers the central opening 68, which is its default position (best see in FIGS. 8 and 9). FIG. 8 shows the central valve 66 covering the central opening 68, and FIG. 9 shows the central valve 66 lifted up so that the central opening 68 is uncovered. On each side of the breathing mask 32, there is a plurality of exhalation holes 36 (shown in FIGS. 7-9). Adjacent to each exhalation hole 36 is a moveable exhalation hole valve 38 (shown in FIGS. 4 and 6), which in its default position covers the exhalation hole 36. The breathing mask 32 also includes a strap 40 for securing the breathing mask 32 to a user.

As shown in FIG. 1, the breathing device 10 also includes tubing 16 having a first end 46 and a second end 48 and a mask tubing connector 34. As shown in FIG. 5, the mask tubing connector 34 is affixed at one end within the central opening 68 of the breathing mask 32 and at the other end to the first end 46 of the tubing 16. The second end 48 of the tubing 16 is inserted into the breathing air bag 42 by inserting the second end 48 of the tubing 16 into the tubing insert 20, the top end 28 of the tubing insert connector 26 and through the split valve 30.

Referring generally to FIGS. 1-9, in use, the breathing mask 32 is placed over the nose and mouth of a user and is secured to the user's head with the strap 40. When the user inhales, the moveable central valve 66 moves upwardly and the central opening 68 is uncovered. This allows air from the breathing air bag 42 to enter the tubing 16 and then enter the breathing mask 32 so that the user inhales the air in the breathing air bag 42, which is either fresh air or varying high concentrations of oxygen with or without aroma. During inhalation, the exhalation holes 36 remain covered by the exhalation hole valves 38. When the user exhales, the moveable central valve 66 moves downwardly and covers the central opening 68, which stops the entry of air from the breathing air bag 42 into the breathing mask 32, and the exhalation hole valves 38 move outwardly to uncover the exhalation holes 36 so that the exhaled air is expelled through the exhalation holes 36.

This pattern of inhaling and exhaling of air by the user is repeated until all of the air in the breathing air bag air 42 is released. It is contemplated that the amount of fresh air or oxygen in the breathing air bag 42 will accommodate about 10 minutes to about 20 minutes of breathing.

Additionally, as the breathing air bag 42 decreases in volume due to the release of air within the air bag 42, air enters the air container 12 via the plurality of air release holes 22 located in the inner cap 18, which prevents formation of a vacuum in the air container 12 as air is released from the breathing air bag 42.

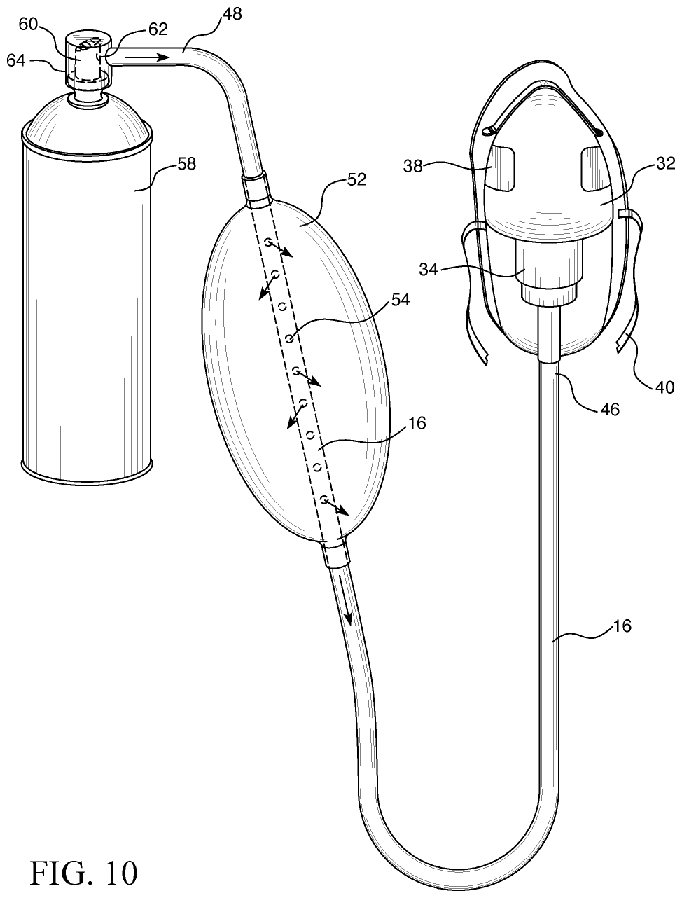

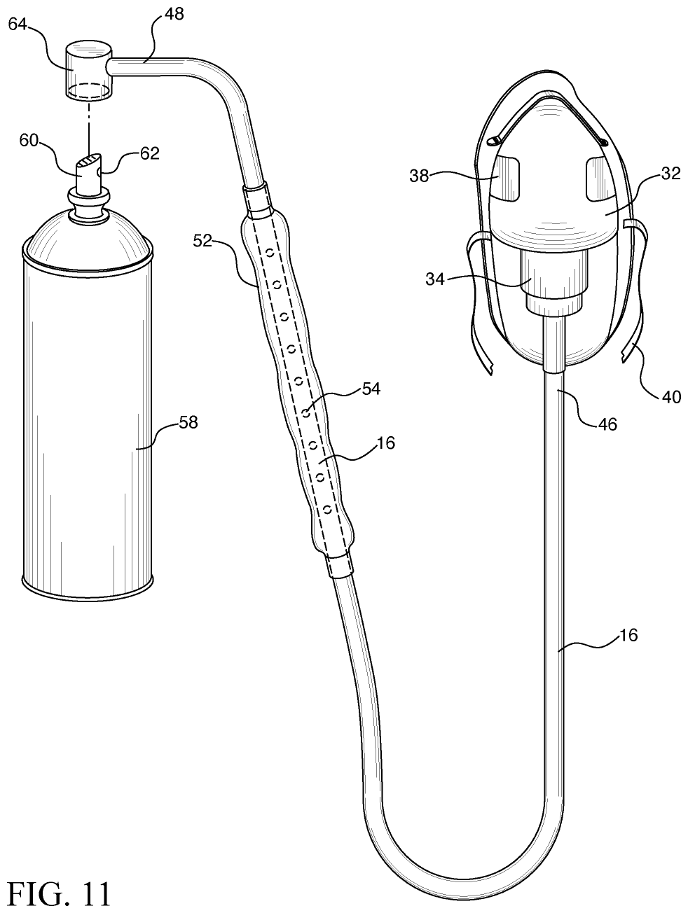

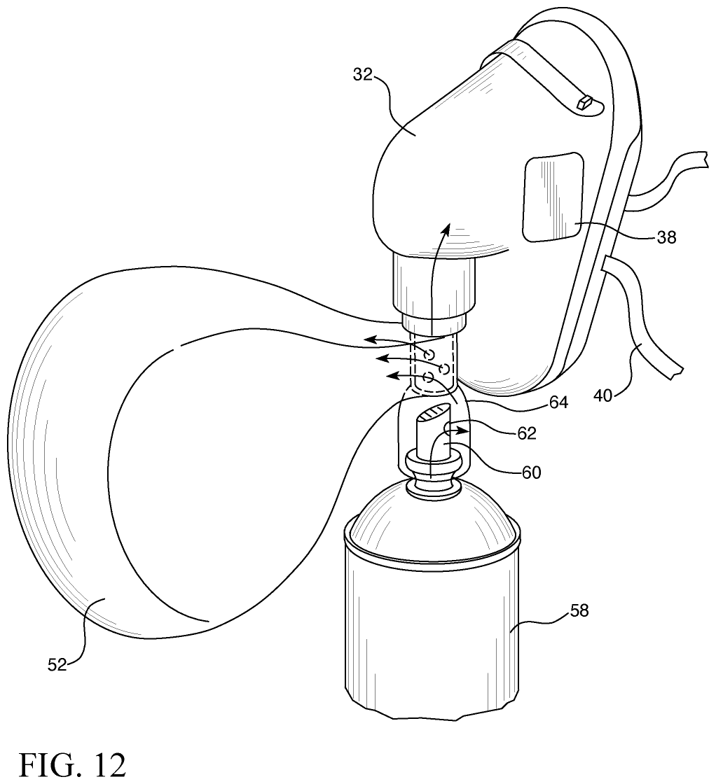

In another embodiment of the invention, as shown in FIGS. 10 and 11, the portable breathing device 10 for delivering fresh air or varying high concentrations of oxygen with or without aroma to a user comprises a can of compressed fresh air or compressed oxygen 58. The can 58 on its top surface includes a release button 60 having a release button air hole 62. Covering the release button 60 is a compressible release hood 64 which has an opening therethrough.

This embodiment of the portable breathing device 10 includes a breathing mask 32 having a central opening 68, a moveable central valve 66 which covers the central opening, a plurality of exhalation holes 36 on each side of the breathing mask 32, a pair of moveable exhalation hole valves 38, each one of the pair adjacent to the exhalation holes 36 in the breathing mask 32 to cover the exhalation holes 36, and a strap 40 to secure the breathing mask 32 to a user. This embodiment also includes tubing 16 having a first end 46 and a second end 48 and a mask tubing connector 34 affixed to the central opening 68 of the breathing mask 32, in which the mask tubing connector 34 is affixed to the first end 46 of the tubing 16, and the second end 48 of the tubing 16 is affixed into the opening of the compressible release hood 64.

This embodiment also comprises an inflatable air reservoir 52 positioned on and encircling a length of the tubing 16, in which the tubing 16 that is encircled by the inflatable air reservoir 52 has a plurality of tubing air holes 54 to allow air to enter and exit the inflatable air reservoir 52.

Referring now to FIG. 10, in use, when a user presses down on the compressible release hood 64, it pushes down on the release button 60 and compressed air is released through the release button air hole 62 into the tubing 16 and exits through the tubing air holes 54 and into the air reservoir 52, which inflates the air reservoir 52. Referring now to FIG. 11, when the breathing mask 32 is placed over the nose and mouth of a user and the user inhales, the moveable central valve 66 moves upwardly to uncover the central opening 68, which pulls the air contained in the air reservoir 52 through the rest of the tubing and into the breathing mask 32 so that the user inhales the fresh air or the varying concentrations of oxygen with or without aroma contained in the air reservoir 52, which then becomes less inflated. When the user exhales, the moveable central valve 66 moves downwardly to cover the central opening 68, which stops the entry of air remaining in the air reservoir 52 and the rest of the tubing 16 into the breathing mask 32, and the exhalation hole valves 38 move outwardly to uncover the exhalation holes 36 so that the exhaled air is expelled through the exhalation holes 36. This pattern of inhaling and exhaling of air by the user is repeated until all of the air in the air reservoir 52 is released. The amount of time that the breathing device may be used varies on how much compressed air is consumed at any one time by the user.

In still another embodiment of the invention, as shown in FIG. 12, the breathing device 10 for delivering fresh air or varying concentrations of oxygen with or without aroma to a user comprises a can of compressed fresh air or compressed oxygen 58 optionally with aroma, in which the can 58 has a release button 60 containing a release button air hole 62 on its top surface. A compressible release hood 64 which has an opening on its top surface covers the release button 60.

This embodiment of the breathing device 10 includes a breathing mask 32 having a central opening 68, a moveable central valve 66 which covers the central opening, a plurality of exhalation holes 36 on each side of the breathing mask 32, a pair of moveable exhalation hole valves 38, each one of the pair adjacent to the exhalation holes 36 in the breathing mask 32 covering the exhalation holes 36, and a strap 40 to secure the breathing mask 32 to a user. A hollow mask interface connector 70 is affixed at one end to the central opening 68 in the breathing mask 32. The other end of the mask interface connector 70 is affixed to one end of a hollow air interface connector 78 having a plurality of air holes therein. The other end of the air interface connector 78 is affixed atop the opening of the compressible release hood 64. An expandable air bag 82 having an opening therein is affixed around the air interface connector 78 by having the opening of the expandable air bag 82 tightly affixed and encircling the air interface connector 78.

In use, when a user presses down on the compressible release hood 64, it pushes down on the release button 60 and compressed air is released through the release button air hole 62 into the opening of the compressible release hood 64 and into the air interface connector 78. Air then enters the expandable air bag 82 as well as the mask interface connector 70. When a user inhales, the central valve 66 moves upwardly which uncovers the central opening 68 so that air in the mask interface connector 70 enters the breathing mask 32. When the user exhales, the central valve 66 moves downwardly to cover the central opening 68 which stops the entry of air into the breathing mask 32 from the expandable air bag 82, and the exhalation hole valves 38 move outwardly to uncover the exhalation holes 36 so that the exhaled air is expelled through the exhalation holes 36. This pattern of inhaling and exhaling of air by the user is repeated until all of the air in the expandable air bag 82 is released.

In each embodiment of the invention, it is contemplated that suitable, light-weight and durable materials, commonly known and used by those in the art, will be used to manufacture the portable breathing device including, without limitation, plastics such as thermoplastic elastomers (TPE), polyvinyl chloride (PVC) and any other suitable plastics.

While the invention has been particularly shown and described with reference to embodiments described above, it will be understood by those skilled in the art that various alterations in form and detail may be made therein without departing from the spirit and scope of the invention, as defined by the appended claims.

What is claimed is:

1. A portable breathing device for delivering fresh air or varying high concentrations of oxygen with or without aroma to a user, comprising:
an air container having an interior and an opening on its top surface;
an outer cap that fits over the opening of the air container;
a breathing air bag located in the interior of the air container and having an opening at one end, said breathing air bag opening having an air bag tubing connector affixed thereon, wherein the breathing air bag contains fresh air or varying high concentrations of oxygen;
an inner cap having a tubing portal and a plurality of air release holes;
a tubing insert having two ends, in which one end fits into the tubing portal;
a tubing insert connector having a top end and a bottom end, said bottom end comprised of a split valve; said top end of the tubing insert connector fitting into the other end of the tubing insert; said bottom end of the tubing insert connector with the split valve fitting into the air bag tubing connector;
a breathing mask having a central opening, a moveable central valve which covers he central opening, a plurality of exhalation holes on both sides of the mask, a pair of moveable exhalation hole valves, each one of the pair adjacent to the exhalation holes in the breathing mask and covering the exhalation holes, and a strap to secure the breathing mask to an individual;
tubing having a first end and a second end;
a mask tubing connector which is affixed at one end within the central opening of the breathing mask and at the other end to the first end of the tubing, said second end of the tubing inserted into the breathing air bag via the tubing insert, the tubing insert connector and the breathing air bag tubing connector.

2. The portable breathing device according to claim 1, wherein the fresh air comprises about 21% oxygen; about 78% nitrogen; about 0.9% argon; about 0.03% carbon dioxide; about 0.002% neon; 0.001% helium; about 0.0002% methane; about 0.0001%; krypton; about 0.0001% hydrogen; about 0.00001% xenon; and a humidity factor ranging between 40% and 60%.

3. The portable breathing device according to claim 1, wherein the varying high concentrations of oxygen range from about 40% to about 100%.

4. The portable breathing device according to claim 1, wherein the fresh air or the varying concentrations of oxygen optionally has an aroma from one or more essential oils contained in the fresh air or the varying concentrations of oxygen.

5. The portable breathing device according to claim 4, wherein the essential oils comprise tangerine, cardamom, bergamot, ginger, caraway, roman chamomile, spearmint, peppermint, lavender and eucalyptus to stimulate appetite; cinnamon, eucalyptus, frankincense, fresh basil to boost mood; and lavender, lemon balm, jasmine, rose, chamomile, bergamot, orange and rosemary to alleviate stress and anxiety, wherein one or more drops of the essential oils is contained in the fresh air or the varying concentrations of oxygen, wherein 1 drop to about 50 drops or higher per essential oil is added to the fresh air or varying high concentrations of oxygen, depending on the needs and/or wishes of the user.

6. The portable breathing device according to claim 1, wherein when the mask is placed over the nose and mouth of a user and the user inhales, the moveable central valve moves upwardly to uncover the central opening, which allows air to enter the mask so that the user inhales the fresh air or the oxygen contained in the breathing air bag; and when the user exhales, the moveable central valve moves downwardly to cover the central opening which stops the entry of air from the breathing air bag into the mask, and the exhalation hole valves move outwardly to uncover the exhalation holes so that the exhaled air is expelled through the exhalation holes.

7. The portable breathing device according to claim 1, wherein the air container is light-weight, rectangular, and has a handle thereon for easy carrying by a user.

8. A method of delivering fresh air or varying high concentrations of oxygen with or without aroma to a user using the device of claim 1, the method comprising:
(a) removing the outer cap from the top of the breathing container;
(b) placing the end of the tubing into the breathing air bag by inserting the tubing through the tubing insert, the tubing insert connector, the split valve and the breathing air bag tubing connector;
(c) placing the breathing mask over the nose and mouth of a user and using the strap to secure the breathing mask to the user's face;
(d) having the user inhale, which moves the central valve upwardly to uncover the central opening, which releases air from the breathing air bag into the tubing and then into the mask so that the user inhales the fresh air or the oxygen contained in the breathing air bag;
(e) having the user exhale, which moves the central valve downwardly to cover the central opening which stops the entry of air from the breathing air bag into the tubing and into the mask, and moves the exhalation hole valves outwardly to uncover the exhalation holes so that the exhaled air is expelled through the exhalation holes;
(f) repeating steps (d) and (e) until all the fresh air or oxygen is released from the breathing bag;
(g) removing the tubing from the breathing air bag and the mask from the user; and
(h) placing the outer cap on the top of the air container.

9. A portable breathing device for delivering fresh air or varying high concentrations of oxygen with or without aroma to a user, comprising:
a can containing compressed fresh air or varying high concentrations of compressed oxygen with or without aroma, said can having on its top surface a release button, said release button containing a release button air hole, and a compressible release hood which covers the release button, said compressible release hood having an opening therethrough;
a breathing mask having a central opening, a moveable central valve which covers the central opening, a plurality of exhalation holes on both sides of the mask, a pair of moveable exhalation hole valves, each one of the pair adjacent to the exhalation holes in the breathing mask to cover the exhalation holes, and a strap to secure the breathing mask to an individual;
tubing having a first end and a second end;
a mask tubing connector affixed to the central opening of the breathing mask, said mask tubing connector affixed to the first end of the tubing, said second end of the tubing affixed into the opening of the compressible release hood; and
an inflatable air reservoir positioned on and encircling a length of the tubing, wherein the tubing that is encircled by the inflatable air reservoir has a plurality of tubing air holes to allow air to enter and exit the air reservoir.

10. The portable breathing device of claim 9, wherein the can of compressed air contains about 21% oxygen; about 78% nitrogen; about 0.9% argon; about 0.03% carbon dioxide; about 0.002% neon; 0.001% helium; about 0.0002% methane; about 0.0001%; krypton; about 0.0001% hydrogen; about 0.00001% xenon; and a humidity factor ranging between 40% and 60%; and the varying high concentrations of oxygen contained in the can of compressed air range from about 40% to about 100%.

11. The portable breathing device according to claim 9, wherein the fresh air or the varying concentrations of oxygen optionally has an aroma from one or more essential oils contained in the fresh air or the varying concentrations of oxygen.

12. The portable breathing device according to claim 11, wherein the essential oils comprise tangerine, cardamom, bergamot, ginger, caraway, roman chamomile, spearmint, peppermint, lavender and eucalyptus to stimulate appetite; cinnamon, eucalyptus, frankincense, fresh basil to boost mood; and lavender, lemon balm, jasmine, rose, chamomile, bergamot, orange and rosemary to alleviate stress and anxiety, wherein one or more drops of the essential oils is contained in the fresh air or the varying concentrations of oxygen, wherein 1 drop to about 50 drops or higher per essential oil is added to the fresh air or varying high concentrations of oxygen, depending on the needs and/or wishes of the user.

13. The portable breathing device according to claim 9, wherein when the compressible release hood is pressed downwardly, compressed air is released into the tubing and exits through the tubing air holes and into the air reservoir, which inflates the air reservoir.

14. The portable breathing device according to claim 9, wherein when the mask is placed over the nose and mouth of a user and the user inhales, the moveable central valve moves upwardly to uncover the central opening, which pulls the air in the air reservoir through the rest of the tubing and into the mask so that the user inhales the fresh air or the oxygen contained in air reservoir; and wherein when the user exhales, the moveable central valve moves downwardly to cover the central opening, which stops the entry of air from the reservoir into the rest of the tubing, and the exhalation hole valves move outwardly to uncover the exhalation holes so that the exhaled air is expelled through the exhalation holes.

15. A method of delivering fresh air or varying high concentrations of oxygen with or without aroma to a user using the device of claim 9, the method comprising:
(a) placing the compressible release hood with affixed tubing over the release button of the can of compressed fresh air or oxygen;
(b) placing the breathing mask over the nose and mouth of a user and using the strap to secure the breathing mask to the user's face;
(c) pressing downwardly on the compressible release hood to release gas into the tubing and into the inflatable air reservoir so that the air reservoir inflates;
(d) having the user inhale, which moves the central valve upwardly to uncover the central opening, which pulls air from the air reservoir into the tubing and then into the mask so that the user inhales the fresh air or the oxygen contained in the can;
(e) having the user exhale, which moves the central valve downwardly to cover the central opening, which stops the entry of air from the air reservoir into the tubing and into the mask, and moves the exhalation hole valves outwardly to uncover the exhalation holes so that the exhaled air is expelled through the exhalation holes; and
(f) repeating steps (d) and (e) until all the fresh air or oxygen is released from the air reservoir.

16. A portable breathing device for delivering fresh air or varying high concentrations of oxygen with or without aroma to a user, comprising:
a can containing compressed fresh air or varying high concentrations of compressed oxygen, said can having on its top surface a release button, said release button containing a release button air hole, and a compressible release hood which covers the release button, said compressible release hood having an opening thereon;
a breathing mask having a central opening in which one end of a hollow mask interface connector is affixed therein, a moveable central valve which covers the central opening, a plurality of exhalation holes on both sides of the mask, a pair of moveable exhalation hole valves, each one of the pair adjacent to the exhalation holes in the breathing mask covering the exhalation holes, and a strap to secure the breathing mask to an individual;
a hollow air interface connector having a first end and second end and a plurality of air holes therein, said first end affixed atop the opening of the compressible release hood, said second end affixed to the other end of the mask interface connector; and
an expandable air bag having an opening therein, said opening of the expandable air bag affixed to and encircling the air interface connector.

17. The portable breathing device of claim 16, wherein the can of compressed air contains about 21% oxygen; about 78% nitrogen; about 0.9% argon; about 0.03% carbon dioxide; about 0.002% neon; 0.001% helium; about 0.0002% methane; about 0.0001%; krypton; about 0.0001% hydrogen; about 0.00001% xenon; and a humidity factor ranging between 40% and 60%; and the varying high concentrations of oxygen contained in the can of compressed air range from about 40% to about 100%.

18. The portable breathing device according to claim 16, wherein the fresh air or the varying concentrations of oxygen optionally has an aroma from one or more essential oils contained in the fresh air or the varying concentrations of oxygen.

19. The portable breathing device according to claim 18, wherein the essential oils comprise tangerine, cardamom, bergamot, ginger, caraway, roman chamomile, spearmint, peppermint, lavender and eucalyptus to stimulate appetite; cinnamon, eucalyptus, frankincense, fresh basil to boost mood; and lavender, lemon balm, jasmine, rose, chamomile, bergamot, orange and rosemary to alleviate stress and anxiety, wherein one or more drops of the essential oils is contained in the fresh air or the varying concentrations of oxygen, wherein 1 drop to about 50 drops or higher per essential oil is added to the fresh air or varying high concentrations of oxygen, depending on the needs and/or wishes of the user.

20. A method of delivering fresh air or varying high concentrations of oxygen with or without aroma to a user using the device of claim 16, the method comprising:
(a) placing the compressible release hood with affixed tubing over the release button of the can of compressed fresh air or varying high concentrations of oxygen with or without aroma;
(b) placing the breathing mask over the nose and mouth of a user and using the strap to secure the breathing mask to the user's face;
(c) pressing downwardly on the compressible release hood to release gas into the air interface connector and into the expandable air bag through the air holes in the air interface connector;
(d) having the user inhale, which moves the central valve upwardly to uncover the central opening which releases air from the expandable air bag into the mask so that the user inhales the fresh air or the oxygen contained in the can;
(e) having the user exhale, which moves the central valve downwardly to cover the central opening which stops the entry of air into the mask from the expandable air bag, and moves the exhalation hole valves outwardly to uncover the exhalation holes so that the exhaled air is expelled through the exhalation holes; and
(f) repeating steps (d) and (e) until all the fresh air or oxygen is released from the expandable air bag.
